(19) United States
(12) Patent Application Publication
(10) Pub. No.: US 2016/0220581 A1
(43) Pub. Date: Aug. 4, 2016

(54) CHROMIUM COMPOSITIONS FOR THE TREATMENT OR PREVENTION OF DIABETIC RETINOPATHY

(71) Applicant: JDS Therapeutics, LLC, Purchase, NY (US)

(72) Inventors: James R. Komorowski, Trumbull, CT (US); Kazim Sahin, Elazig (TR)

(21) Appl. No.: 15/010,395

(22) Filed: Jan. 29, 2016

Related U.S. Application Data
(63) Continuation of application No. 62/110,234, filed on Jan. 30, 2015.

(51) Int. Cl. A61K 31/555; A61K 38/28; A61K 31/155; A61K 9/00

(52) U.S. Cl. CPC A61K 31/555; A61K 9/0048; A61K 38/28; A61K 31/155

(57) ABSTRACT

The embodiments discloses herein related chromium compositions and methods of using same for the treatment and/or prevention of diabetic retinopathy. The present application is based, in part, on the surprising discovery that the administration of chromium complexes and, in particular, the administration of chromium histidinate, improves diabetic retinopathy and symptoms thereof, reduces the levels of retina malondialdehyde and glycosylated hemoglobin, and decreases oxidative stress and lipid oxidation in the eye/retina.

### CROSS-REFERENCE TO RELATED APPLICATIONS

This application claims the benefit of U.S. Provisional Application No. 61/110,234, filed Jan. 30, 2015; which is incorporated herein by reference in its entirety, including any drawings.

### BACKGROUND

1. Field

The embodiments discloses herein related chromium compositions and methods of using same for the treatment and/or prevention of diabetic retinopathy. The present application is based, in part, on the surprising discovery that chromium histidinate (“CrHis”) can be used to treat and/or prevent diabetic retinopathy. CrHis may also be used to reduce levels of retina malondialdehyde and glycosylated hemoglobin and/or decrease oxidative stress and lipid oxidation in the retina.

2. Description of Related Art

### Diabetic Retinopathy

Diabetic retinopathy is a common diabetic eye disease and a leading cause of blindness in working-age population. The pathophysiology of diabetic retinopathy is complex and multifactorial. The pathogenic process involves intricate interactions between oxidative stress and hyperglycemia.

In a high energy-demanding tissue such as retina, the regulation of glucose uptake and its utilization is important for the maintenance of normal retinal function. Glucose uptake is regulated by glucose transporter proteins (GLUTs) and all mammalian cells contain one or more members of this GLUT protein family. Glucose uptake into retina cells occurs across the blood-retinal barrier.

### Chromium

Chromium is an essential trace element. The essentiality of chromium in the diet was established in 1959 by Schwartz. (Schwartz, “Present Knowledge in Nutrition,” page 571, fifth edition (1984, the Nutrition Foundation, Washington, D.C.)). Chromium is essential for optimal insulin activity in all known insulin-dependent systems (Boyle et al. (1977) Southern Med. J. 70:1449-1453). Chromium depletion is characterized by the disturbance of glucose, lipid and protein metabolism and by a shortened lifespan. Insufficient dietary chromium has been linked to both maturity-onset diabetes and to cardiovascular disease.

Dietary supplementation of chromium to normal individuals has been reported to lead to improvements in glucose tolerance, serum lipid concentrations, including high-density lipoprotein cholesterol, insulin and insulin binding. (Anderson (1986) Clin. Psychol. Biochem. 4:31-41). Supplemental chromium in the trivalent form, e.g. chromic chloride, is associated with improvements of risk factors associated with adult-onset (Type 2) diabetes and cardiovascular disease. Chromium supplementation has been shown to reduce hyperglycemia, as well as promote weight loss, as described in U.S. Pat. Nos. 5,929,066, 6,329,361, and 6,809,115, which are each hereby incorporated by reference in their entirety. In a clinical study, Anderson et al. (Metabolism (1987) 36(4):351-355, 1987), chromium supplementation was shown to alleviate hypoglycemic symptoms and raise serum glucose levels out of the hypoglycemic range. In another study, chromium supplementation to overweight children with Type 1 diabetes did not result in any cases of hypoglycemia (May, 2007). In yet another study, chromium supplementation to adults with Type 1 diabetes did not result in any cases of hypoglycemia; and allowed a 50% reduction in insulin dose (Ravina et al. (1995) J. Trace Elements in Experimental Med. 12:71-83).

The principal energy sources for the body are glucose and fatty acids. Chromium depletion results in biologically ineffective insulin and compromised glucose metabolism. Under these conditions, the body relies primarily upon lipid metabolism to meet its energy requirements, resulting in the production of excessive amounts of acetyl-CoA and ketone bodies. Some of the acetyl-CoA can be diverted to increased cholesterol biosynthesis, resulting in hypercholesterolemia. Diabetes mellitus is characterized in large part by glycosuria, hypercholesterolemia, and often ketoacidosis. The accelerated atherosclerotic process seen in diabetics is associated with hypercholesterolemia. (Boyle et al. (1977) Southern Med. J. 70:1449-1453).

Chromium functions as a cofactor for insulin. It binds to the insulin receptor and potentiates many, and perhaps all, of its functions. (Boyle et al. (1977) Southern Med. J. 70:1449-1453). These functions include, but are not limited to, the regulation of carbohydrate and lipid metabolism. (Schwartz, “Present Knowledge in Nutrition,” page 571, fifth edition (1984, the Nutrition Foundation, Washington, D.C.)). The introduction of inorganic chromium compounds per se into individuals is not particularly beneficial. Chromium must be converted endogenously into an organic complex or must be consumed as a biologically active molecule. Only about 0.5% of ingested inorganic chromium, however, is assimilated into the body. (Recommended Daily Allowances, Ninth Revised Edition, The National Academy of Sciences, page 160, 1980). Only 1-2% of most organic chromium compounds are assimilated into the body.

U.S. Pat. Nos. 4,315,927 and Re. 33,988 disclose that when selected essential metals, including chromium, are administered to mammals as exogenously synthesized coordination complexes of picolinic acid, they are directly available for absorption without competition from other metals. Describes therein are compositions and methods for selectively supplementing the essential metals in the human diet and for facilitating absorption of these metals by intestinal cells. These complexes are safe, inexpensive, biocompatible, and easy to produce. The exogenously synthesized essential metal coordination complexes of picolinic acid (pyridine-2-carboxylic acid) have the following structural formula:

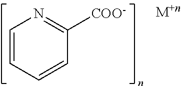

wherein M represents the metallic cation and n is equal to the cation's valence. For example, when M is Cr and n=3, then the compound is chromic tripicolinate. Other chromium picolinates disclosed include chromic monopicolinate and chromic dipicolinate.

The U.S. Recommended Daily Intake (RDI) of chromium is 120 μg. U.S. Pat. No. 5,087,623, the entire contents of which are hereby expressly incorporated herein by reference, describes the administration of chromic tripicolinate for the treatment of adult-onset diabetes in doses ranging from 50 to 500 μg. U.S. Pat. No. 6,329,361, the entire contents of which are hereby expressly incorporated herein by reference, discloses the use of high doses of chromic tripicolinate (providing 1,000-10,000 μg chromium/day) for reducing hyperglycemia and stabilizing the level of serum glucose in humans with Type 2 diabetes. U.S. Pat. Nos. 5,789,401 and 5,929,066, the entire contents of which are hereby expressly incorporated herein by reference, disclose a chromic tripicolinate-biotin composition and its use in lowering blood glucose levels in humans with Type 2 diabetes.

U.S. Pat. Nos. 5,087,623; 5,087,624; and 5,175,156, the entire contents of which are hereby expressly incorporated herein by reference, disclose the use of chromium tripicolinate for supplementing dietary chromium, reducing hyperglycemia and stabilizing serum glucose, increasing lean body mass and reducing body fat, and controlling serum lipid levels, including the lowering of undesirably high serum LDL-cholesterol levels and the raising of serum High Density Lipid (HDL)-cholesterol levels. U.S. Pat. Nos. 4,954,492 and 5,194,615, the entire contents of which are hereby expressly incorporated by reference, describe a related complex, chromic nicotinate, which is also used for supplementing dietary chromium and lowering serum lipid levels. Picolinic acid and nicotinic acid are position isomers having the following structures:

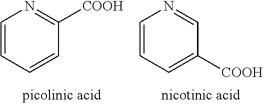 [SMILES: O=C(O)c1ccccn1 ; O=C(O)c1cccnc1]

Nicotinic acid and picolinic acid form coordination complexes with monovalent, divalent and trivalent metal ions and facilitate the absorption of these metals by transporting them across intestinal cells and into the bloodstream. Chromium absorption in rats following oral administration of CrCl₃ was facilitated by the non-steroidal anti-inflammatory drugs (NSAIDs) aspirin and indomethacin. (Davis et al. (1995) J. Nutrition Res. 15:202-210; Kamath et al. (1997) J. Nutrition 127:478-482). These drugs inhibit the enzyme cyclooxygenase which converts arachidonic acid to various prostaglandins, resulting in inhibition of intestinal mucus formation and lowering of intestinal pH which facilitates chromium absorption.

There remains a constant need for effective treatments of diabetic retinopathy and associated conditions. The present embodiments disclosed herein address this need by providing a safe, inexpensive, drug-free therapeutic agent, and methods of administering the same. Different forms of chromium exhibit different and unpredictable absorption and activity profiles in vivo. In order for a chromium complex to exert a biological effect in vivo, it must (1) be absorbed by the body; (2) be absorbed in the right cells (tissues or organs); and (3) must be released from the complex once within the cells. The fact that a particular chromium complex may be absorbed by certain cells does not necessarily guarantee a biological effect. Whether a particular form of chromium will be effectively absorbed, let alone whether the chromium will be released from the complex once absorbed to exert any physiological effect is unpredictable.

### SUMMARY

The embodiments disclosed herein are based, in part, upon the surprising discovery that chromium and histidinate, chromium histidinate complexes, and combinations thereof possess improved therapeutic efficacy and benefits in treating and/or preventing diabetic retinopathy and associated conditions. Chromium histidinate complexes may have a greater therapeutic effect than other chromium complexes when used to treat or prevent diabetic retinopathy and its associated symptoms. See, e.g., U.S. Patent Appl. No. 2010/0009015, incorporated by reference in its entirety.

Embodiments disclosed herein relate to the use of compositions comprising, consisting essentially of, or consisting of chromium and histidine, chromium histidinate complexes, chromium trihistidinate, chromium polyhistidinate complexs, or combinations thereof, including pharmaceutically acceptable salts, hydrates, solvates, or mixtures thereof for the improved treatment and/or prevention of diabetic retinopathy and related conditions, diseases, and disorders.

Some embodiments comprise methods of treating and/or preventing and/or ameliorating the symptoms of diabetic retinopathy by administering chromium histidinate, chromium trihistidinate, or chromium polyhistidinate, or any combination thereof, to a patient. Some embodiments comprise methods of lowering the levels of retina malondialdehyde by administering chromium histidinate, chromium trihistidinate, or chromium polyhistidinate, or any combination thereof, to a patient in need thereof. Some embodiments comprise methods of lowering the levels of glycosylated hemoglobin by administering chromium histidinate, chromium trihistidinate, or chromium polyhistidinate, or any combination thereof, to a patient in need thereof. Some embodiments comprise methods of decrease treating and/or preventing and/or reducing oxidative stress in the retina by administering chromium histidinate, chromium trihistidinate, or chromium polyhistidinate, or any combination thereof, to a patient in need thereof. Some embodiments comprise methods of decrease treating and/or preventing and/or reducing lipid oxidation in the retina by administering chromium histidinate, chromium trihistidinate, or chromium polyhistidinate, or any combination thereof, to a patient in need thereof.

Some embodiments relate to decreasing metabolic abnormalities implicated in the pathogenesis of diabetic retinopathy. In some embodiments, these metabolic abnormalities comprise increased oxidative stress, increased lipid peroxidation, hyperglycemia, and increased protein glycation. Some embodiments relate to reducing free radical oxidation of the retinal photoreceptors. Some embodiments relate to decreasing or preventing loss of lipoprotein membrane content. Some embodiments relate to decreasing or preventing the retinal capillary basement membrane from thickening. Some embodiments relate to decreasing or preventing retinal microangiopathy.

Some embodiments relate to decreasing the risk of loss in visual acuity in individuals having diabetic retinopathy. Some embodiments relate to preventing loss in visual acuity in individuals having diabetic retinopathy. Some embodiments relate to reducing the degree of retinal hard exudates. Some embodiments relate to decreasing ocular glucose levels. Some embodiments relate to decreasing ocular cholesterol levels. Some embodiments relate to decreasing ocular triglyceride levels. Some embodiments relate to decreasing levels of glycosylated hemoglobin.

Some embodiments relate to pharmaceutical compositions comprising one or more compositions disclosed herein, with a pharmaceutically acceptable vehicle, excipient, or diluent. For example, pharmaceutically acceptable vehicles can include carriers, excipients, diluents, and the like, as well as combinations or mixtures thereof.

Some embodiments relate to co-administration with antidiabetic drugs to provide an additive effect. Some embodiments relate to co-administration with antidiabetic drugs to provide a synergistic effect. Some embodiments relate to improving an individual's carbohydrate metabolic profile. In some embodiments, a carbohydrate metabolic profile is assessed by measuring expression of insulin as well as various glucose transporter proteins. Some embodiments relate to improving insulin binding to retinal cells.

In some aspects, the effective amount of chromium in the composition can be between about 5 and 2,000 micrograms. In some aspects, the chromium is selected from the group of chromium complexes consisting of chromium picolinate, chromic tripicolinate, chromium nicotinate, chromic polynicotinate, chromium chloride, chromium histidinate, chromium trihistidinate, and chromium yeasts. Preferably, the chromium comprises chromium histidinate. In some aspects, the composition comprises chromium histidinate in combination with one or more additionally chromium complexes.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In some embodiments, the composition is topically administered to the eye. Some embodiments relate to a solid formulation. Other modes of administration useful in the methods include but are not limited to intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin.

In some aspects, a method for treating, preventing, or ameliorating diabetic retinopathy in a subject in need thereof, comprises identifying a subject having or at risk for developing diabetic retinopathy and administering a therapeutically effective amount of at least one chromium complex. The at least one chromium complex may consists essentially of chromium and histidine, a chromium histidinate complex, or combinations thereof. At least one chromium complex may be co-administered with a second therapeutic agent selected from the group consisting of insulin, metformin, and a chromium-insulin complex. The second therapeutic agent may be administered orally. The administering a therapeutically effective amount of at least one chromium complex may comprise administering a topical ophthalmic formulation.

In some aspects, a formulation for topical ophthalmic administration comprises a therapeutically effective amount of one or more chromium complexes and at least one ophthalmically acceptable excipient. The formulation may include a second therapeutic agent selected from the group consisting of insulin, metformin, and a chromium-insulin complex.

### BRIEF DESCRIPTION OF THE FIGURES

FIGS. 1A-1E illustrates the effect of chromium supplementation on the expression of, GLUT 1 (Panel A), GLUT3 (Panel B), insulin (Panel C), MDA (Panel D) values, and density of proteins (Panel E) in the retina tissue regions of DR rats. Data are means of quadruplets of assays and expressed as relative to control (%). Blots were repeated at least three times (n=3) and a representative blot for each is shown. Actin was included to ensure equal protein loading. Values are LS means±SE. Different letters within the retina parts indicate statistical differences among groups (p<0.05).

FIG. 2 illustrates paraffin section photograph(s) of rat retina control and experimental group, control (A), CrHis alone (B), STZ+CrHis (C), and STZ alone (D) showing the histopathological changes.

### DETAILED DESCRIPTION

### Chromium

As used herein, the term “chromium” refers to chromium chloride, chromium yeasts, as well as chromium complexes. Some chromium complexes useful in the embodiments disclosed herein include, but are not limited to, the following: chromium histidinate; chromium trihistidinate; chromium polyhistidinate; chromium dinicocysteinate; chromium dinicotinate tryptophan; chromium dinicotinate tyrosine; chromium dinicotinate hydroxycitrate; chromium dinicotinate cinnamate; chromium dinicotinate gallate; chromium dinicotinate 5-hydroxytryptophan; chromium dinicotinate aspartate; chromium dinicotinate glutamate; chromium dinicotinate arginate; chromium tris(tryptophan); chromium nicotinate, chromium polynicotinate; chromium picolinate; chromium monopicolinate; chromium dipicolinate; chromium tripicolinate; chromium triphenylalanine; chromium tris(tyrosine); chromium tris(hydroxycitrate); chromium tris(5-hydroxytryptophan); chromium tris(cinnamate); chromium tris(gallate); chromium complexes disclosed herein are chromium having three different carboxylate ligands.

As used herein, the term “hydrophilic chromium complex” or “fast acting chromium complex” refers to a chromium complex that is charged at physiological pH, or has polar properties. Non-limiting examples of hydrophilic, fast-acting chromium complexes include chromium acetate, chromium chloride, chromium histidinate and chromium nicotinate, and the like, or any pharmaceutically acceptable salts, hydrates, solvates, or mixtures thereof.

The term “lipophilic chromium complex” or “slow-acting chromium complex” refers to a chromium complex that is not charged at physiological pH, and that does not have polar properties. Chromium picolinate, and any pharmaceutically acceptable salts, hydrates, or solvates thereof, is a non-limiting example of a lipophilic, slow-acting chromium complex.

The eye includes multiple parts, such as the aqueous humor, vitreous humor, and the retina. One skilled in the art would recognize that references to the “eye” and to the “retina” may include overlapping parts of the eye.

In preferred embodiments, the hydrophilic chromium complex or the “fast-acting” chromium complex is chromium histidinate, chromium trihistidinate, or chromium polyhistidinate, or any combination thereof. Preferably, the lipophilic chromium complex or the “slow-acting” chromium complex is chromium picolinate.

In various cases, the ligand(s) has/have the ability to bond to chromium via its carboxylate functional group as well as through pi electron-d orbital interaction. This secondary interaction between the ligand and chromium can increase the bioavailability and absorption of chromium.

In some embodiments, the chromium can be in the form of complexes of trivalent chromium and at least one and no more than three tyrosine or tryptophan ligands. In specific embodiments, the chromium can be in the form of chromium complexes such as chromium (III) tris(tryptophan) and chromium (III) tris(tyrosine).

In some embodiments, the chromium complexes can be complexes of trivalent chromium and one or more compounds extracted from plants. Non-limiting examples of plants from which these compounds can be extracted include plants such as genus Garcinia, Groffonia simplicifolia, cinnamon bark, gallnuts, sumac, witch hazel, tea leaves, and oak bark. For example, in some embodiments, chromium can be provided in the form of chromium hydroxycitrate, chromium hydroxytryptophan, chromium cinnamate, and chromium gallate.

Preferably, the chromium is provided as a combination of chromium picolinate and chromium histidinate, or as a combination of chromium nicotinate and chromium histidinate. In other preferred embodiments, the chromium is provided as chromium histidinate. In another preferred embodiment, chromium is provided as a chromium histidinate complex. The compositions disclosed herein may consist of, consist essentially or, and/or comprise chromium histidinate complexes.

While the chromium complexes aid in the absorption of chromium by intestinal cells, in some embodiments, uncomplexed chelating agents are advantageously included in the compositions to facilitate absorption of other ingested chromium as well as other metals including, but not limited to, copper, iron, magnesium, manganese, and zinc. Suitable chelating agents include histidine, any essential amino D or L amino acids, tri amino acid formulae including but not limited to, triphenylalanine, tri histidine, tri arginine, picolinic acid, nicotinic acid, or both picolinic acid and nicotinic acid.

Chelating agents such as histidine, picolinic acid and nicotinic acid are available from many commercial sources, including Sigma-Aldrich (St. Louis, Mo.) (picolinic acid; catalog No. P5503; nicotinic acid; catalog No. PN4126). In some embodiments, the ratio of the chromium complex to the chelating agent in the embodiments disclosed herein can be from about 10:1 to about 1:10 (w/w), more preferably from about 5:1 to about 1:5 (w/w), e.g., 5:1, 5:2, 5:3, 5:4, 1:1; 1:2, 1:3, 1:4, 1:5, or any number in between. Alternatively, the molar ratio of chromium complex to the uncomplexed chelating agent is preferably 1:1, and can be from about 5:1 to about 1:10, e.g., e.g., 5:1, 5:2, 5:3, 5:4, 1:1; 1:2, 1:3, 1:4, 1:5, 1:6, 1:7, 1:8, 1:9, 1:10, or any number in between. The chelating agents with D or L amino acid and or with tri or mono and di forms of chromium complex with tri amino acid or one or more amino acids but not limited to chromium triphenylanine, chromium trihistidine, chromium poly phenylanine, chromium poly hisitidine, chromium polynicotinate, chromium di phenylananine, chromium di picolinic acid, chromium di hisitidine etc.

Some embodiments provide methods of identifying a subject having diabetic retinopathy. Some embodiments provide methods of identifying a subject at risk of developing diabetic retinopathy. In some embodiments, identifying the subject having or at risk of developing diabetic retinopathy comprises performing blood tests, including, but not limited to testing blood glucose levels, malondialdehyde levels, anti-oxidant levels, cortisol levels, insulin levels, oxidative stress markers, oxidized fatty acids, and hemoglobin Alc.

In some embodiments, identifying the subject having or at risk of developing diabetic retinopathy comprises performing eye examinations, including, but not limited to fundus photographic sets (for example, two fundus images from each eye), visual acuity testing, tonometry of the eye(s), pupil dilation and physical examination of the retina, ophthalmoscopy, slit lamp exam, gonioscopy, and optical coherence tomography (OCT). In some embodiments, the subject has symptomatic diabetes. In some embodiments, the subject has asymptomatic diabetes.

Some embodiments provide methods of decreasing levels of malondialdehyde in the eye. In some embodiments, malondialdehyde levels in the eye are decreased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or at least about 99%.

Some embodiments provide methods of decreasing levels of HbAlc in the eye. In some embodiments, HbAlc levels in the eye are decreased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or at least about 99%.

Some embodiments provide methods of decreasing levels of oxidized lipids in the eye. In some embodiments, oxidized lipid levels in the eye are decreased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or at least about 99%.

Some embodiments provide methods of improving visual acuity. In some embodiments, visual acuity is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or at least about 99%, using standard measures of visual acuity.

Some embodiments provide compositions and methods of treating subjects with compositions that comprise or consist of a therapeutically effective amount of chromium. Some embodiments provide compositions and methods of treating subjects with compositions that comprise, consist essentially of, or consist of a therapeutically effective amount of insulin. Some embodiments provide compositions and methods of treating subjects with compositions that comprise, consist essentially of, or consist of a therapeutically effective amount of chromium and a therapeutically effective amount of insulin. For example, some embodiments provide compositions and method of treating subjects that comprises, consists essentially of, or consist of a chromium-insulin complex. Various methods of treatment are discussed below.

A “therapeutically effective amount” as used herein includes within its meaning a non-toxic but sufficient amount of a compound active ingredient or composition comprising the same for use in the embodiments disclosed herein to provide the desired therapeutic effect. The exact amount of the active ingredient disclosed herein required will vary from subject to subject depending on factors such as the species being treated, the age and general condition of the subject, the severity of the condition being treated, the particular agent being administered, the weight of the subject, and the mode of administration and so forth. Thus, it is not possible to specify an exact “effective amount”. However, for any given case, an appropriate “effective amount” may be determined by one of ordinary skill in the art using only routine methods.

By way of example, a “therapeutically effective amount” of the chromium disclosed herein can be, for example, 0.001 μg/kg, 0.01 μg/kg, 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 80 μg/kg 0, 850 μg/kg, 900 μg/kg, 1 mg/kg, 1.5 mg.kg, 2.0 mg/kg, 2.5 mg/kg, 3 mg/kg, 4.0mg/kg, 5.0 mg/kg, 6 mg/kg, 7 mg/kg, 8 mg/kg, 9 mg/kg, 10 mg/kg, 15 mg/kg, 20 mg/kg, 25 mg/kg, 30 mg/kg, 35 mg/kg, 40 mg/kg, 45 mg/kg 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 125 mg/kg, 150 mg/kg, 200 mg/kg, 250 mg/kg, 300 mg/kg, 350 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, lg/kg, 5 g/kg, 10 g/kg, or more, or any fraction in between of chromium. Accordingly, in some embodiments, the dose of chromium in compositions disclosed herein can be about 0.001 μg to about 100 g, preferably per day. For example, the amount of chromium can be 0.001 μg, 0.01 μg, 0.1 μg, 0.2 μg, 0.3 μg, 0.4 μg, 0.5 μg, 0.6 μg, 0.7 μg, 0.8 μg, 0.9 μg, 1 μg, 2 μg, 3 μg, 4 μg, 5 μg, 6 μg, 7 μg, 8 μg, 9 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 125 μg, 150 μg, 175 μg, 200 μg, 225 μg, 250 μg, 275 μg, 300 μg, 325 μg, 350 μg, 375 μg, 400 μg, 425 μg, 450 μg, 475 μg, 500 μg, 525 μg, 575 μg, 600 μg, 625 μg, 650 μg, 675 μg, 700 μg, 725 μg, 750 μg, 775 μg, 800 μg, 825 μg, 850 μg, 875 μg, 900 μg, 925 μg, 950 μg, 975 μg, 1000 μg, 1.25 g, 1.5 g, 1.75 g, 2.0 g, 2.25 g, 2.5 g, 2.75 g, 3.0 g, 3.25 g, 3.5 g, 3.5 g, 3.75 g, 4.0 g, 4.25 g, 4.5 g, 4.75 g, 5.0 g, 5.25 g, 5.5 g, 5.75 g, 6.0 g, 6.25 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75g, 10 g, 20 g, 30 g, 40 g, 50 g, 60 g, 70 g, 80 g, 90 g, 100 g, or more, or any range or amount in between any two of the preceding values. The exemplary therapeutically effective amounts listed above, can, in some embodiments be administered in the methods described elsewhere herein on an hourly basis, e.g., every one, two, three, four, five, six, seven, eight, nine, ten, eleven, twelve, thirteen, fourteen, fifteen, sixteen, seventeen, eighteen, nineteen, twenty, twenty-one, twenty-two, twenty-three hours, or any interval in between, or on a daily basis, every two days, every three days, every four days, every five days, every six days, every week, every eight days, every nine days, every ten days, every two weeks, every month, or more or less frequently, as needed to achieve the desired therapeutic effect.

In some embodiments, the compositions disclosed herein, e.g., compositions that comprise a chromium complex, can be administered to a subject 1 time, 2 times, 3 times, 4 times 5 times, 6 times, 7 times, 8 times, 9 times, 10 times, or more, per day, for a period of time, such as 1 day, 2 days, 3 days, 4 days, 5 days, 6 days, 7 days, 8 days, 9 days, 10 days, 11 days, 12 days, 13 days, 2 weeks, 3 weeks, 4 weeks, 5 weeks, 6 weeks, 7 weeks, 2 months, 3 months, 4 months, 5 months, 6 months, 7 months, 8 months, 9 months, 10 months, 11 months, 1 year, or more, or any amount of time in between the preceding values.

In some embodiments, the compositions described herein, for example compositions that comprise chromium complexes can be administered to a subject per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

By way of example, some embodiments are formulated for topical ophthalmic administration. Some embodiments comprise a sterile solution, a preservative, a solubility enhancer, a viscosity building agent, a surfactant, a pH adjusting agent, a tonicity agent, an antioxidant, or combinations thereof.

Some embodiments comprise a solution for topical ophthalmic administration having a pH from about 3.0 to about 9.0. Some embodiments comprise a solution having a pH from about 4.0 to about 8.0. Some embodiments comprise a solution having a pH from about 4.5 to about 8.0. Some embodiments comprise a solution having a pH from about 5.0 to about 8.0. Some embodiments comprise a solution having a pH from about 5.5 to about 8.0. Some embodiments comprise a solution having a pH from about 6.0 to about 8.0. Some embodiments comprise a solution having a pH from about 6.5 to about 8.0. Some embodiments comprise a solution having a pH from about 7.0 to about 8.0. Some embodiments comprise a solution having a pH from about 7.5 to about 8.0. Some embodiments comprise a solution having a pH from about 6.5 to about 7.5.

Some embodiments comprise a solution for topical ophthalmic administration having an osmolarity of about 150 milliosmoles per kilogram of water (mOsm/kg) to about 450 mOsm/kg. Some embodiments comprise a solution for topical ophthalmic administration having an osmolarity of about 200 mOsm/kg to about 450 mOsm/kg. Some embodiments comprise a solution for topical ophthalmic administration having an osmolarity of about 225 mOsm/kg to about 400 mOsm/kg. Some embodiments comprise a solution for topical ophthalmic administration having an osmolarity of about 250 mOsm/kg to about 375 mOsm/kg. Some embodiments comprise a solution for topical ophthalmic administration having an osmolarity of about 275 mOsm/kg to about 350 mOsm/kg. Some embodiments comprise a solution for topical ophthalmic administration having an osmolarity of about 300 mOsm/kg to about 325 mOsm/kg.

Some embodiments described herein relates to a composition, that can include an effective amount of one or chromium complexes described herein (e.g., CrHis), and a carrier, diluent, excipient or combination thereof.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a composition that lacks biological activity but may be otherwise necessary or desirable. For example and without limitation, it may also be a liquid for the dissolution of a compound to be administered to the eye, and/or by injection, ingestion, or inhalation. A common form of diluent in the art is a buffered aqueous solution such as, without limitation, phosphate buffered saline that mimics the composition of human blood or tears.

As used herein, an “excipient” refers to an inert substance that is added to a composition to provide, without limitation, bulk, consistency, stability, binding ability, lubrication, disintegrating ability etc., to the composition. A “diluent” is a type of excipient.

The compositions described herein can be administered to a human per se, or in compositions where they are mixed with other active ingredients, or carriers, diluents, excipients or combinations thereof. Proper formulation is dependent upon the route of administration chosen. Techniques for formulation and administration of the compounds described herein are known to those skilled in the art.

The compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the combinations disclosed herein may be provided as salts with pharmaceutically compatible counterions.

One may also administer the compound in a local rather than systemic manner, for example, via administering the solution as an eye drop. In some embodiments, the eye drops consist essential of chromium histidinate complexes.

The compositions may, if desired, be presented in a pack or dispenser device which may contain one or more unit dosage forms containing the active ingredient. The pack may for example comprise metal or plastic foil, such as a blister pack, or in single-use eye drop containers. The pack or dispenser device may be accompanied by instructions for administration. Compositions that can include a compound described herein formulated in a compatible carrier may also be prepared, placed in an appropriate container, and labeled for treatment of an indicated condition.

Advantageously, an individual is administered a pharmaceutically effective dose of a chromium complex such as chromium histidinate alone or in combination with at least one other chromium complex. In one embodiment, a composition disclosed herein (e.g., chromium histidinate) and another chromium complex are administered substantially simultaneously. In an alternative embodiment, the compositions disclosed herein (e.g., chromium histidinate) and another chromium complex are provided to the subject sequentially in either order. If administered separately, the chromium complex and diet and composition disclosed herein (e.g., chromium histidinate) should be given in a temporally proximate manner, e.g., within a twenty-four hour period. More particularly, the chromium complex and composition disclosed herein (e.g., chromium histidinate) can be given within one hour of each other.

### EXAMPLES

These examples are provided for illustrative purposes only and not to limit the scope of the claims provided herein.

### General Procedures

Diabetes was induced with streptozotocin [(STZ), 55 mg/kg] by intraperitoneal injection in male Long-Evans rats. Three weeks after STZ injection, rats were divided into four groups, namely, untreated normal controls, normal rats receiving CrHis (110 μg/kg/day); untreated diabetics and diabetics treated with CrHis (110 μg/kg/day) orally for 12 weeks.

In the untreated diabetic group, levels of serum glucose, glycosylated haemoglobin (HbAlc), total cholesterol (TC) and retina malondialdehyde (MDA) were significantly increased, while expressions of retina insulin, and glucose transporter 1 (GLUT 1) and glucose transporter 3 (GLUT3) and level of serum insulin were decreased.

Twenty eight Long Evans rats per experiment, aged 8 weeks with 250±20 g of body weight were used in these experiments. All the animals were kept and maintained under standard guidelines. The animals were kept and maintained at 22±2° C., humidity of 55%±5% and 12/12-hour light/dark cycle. The rats were weighed every week and at the end of the study. Blood sample was collected from the tail vein of each rat for the measurement of biochemical efficacy and safety markers.

The CrHis was given in the water and administered at a concentration of 110 μg/kg bw/d) to get 9.16 μg elemental Cr (kg body/d), which is an equivalent dose of 614 μg Cr for a 70-kg adult human based on previous work. The Cr concentration of the water provided the control group was negligible (<1 μg/L). The water provided the Cr-supplemented group was initially prepared as a solution containing 3000 μg CrHis/L of water. The CrPic-supplemented water was diluted to achieve the target Cr intake per group on the basis of measured water intake. To induce experimental diabetes, STZ was dissolved in citrate buffer (pH 4.5) and injected once intraperitoneally at a dose of 55 mg/kg to the remainder of the animals. A control group was given citrate buffer via intraperitoneal injection.

Fourteen rats were treated with STZ (55 mg/kg body weight) through intraperitoneal injection. All rats were then fasted for 16 hour prior to treatment, but they had access to drinking water. The animals were divided into 4 groups: group I (Control) rats received citrate buffer intraperitoneally and isotonic saline, orally; group II (Control+CrHis) rats were administered chromium histidinate orally (110 μg/kg body weight) daily for a period of four weeks; group III (Diabetic) rats received single injection of STZ (55 mg/kg body weight) intraperitoneally and were also given isotonic saline, orally for the duration of the study; group IV (Diabetic+CrHis) diabetic rats were administered chromium orally as chromium histidinate (110 μg/kg body weight) daily for a period of 12 weeks after the induction of diabetes.

Body weight and blood glucose concentrations were monitored weekly. Blood was collected from the tail vein of the rats. Blood glucose was determined by one touch glucometer (ACCU-Check Active, Roche Diagnostics, Mannheim, Germany) after the injection for 72 h. Before STZ injection, glucose concentrations of study rats and controls were measured and compared. After the injection of STZ, animals that exhibited fasting glucose levels greater than 140 mg/dL were considered as neonatal STZ diabetic resembling diabetes mellitus in humans.

Blood samples were centrifuged at 3000×g for 10 min and sera were separated. Serum glucose concentrations were measured by using ACCU-Chek Active (Roche Diagnostics, Basel, Switzerland). Serum insulin levels were measured with the Rat Insulin Kit (Linco Research, St Charles, Mo.) by enzyme-linked immunosorbent assay (ELISA, Elx-800, BioTek Instruments, Winooski, Vt.). Serum concentrations of total cholesterol (TC) were measured by diagnostic kits (Sigma Diagnostics, St Louis, Mo.). Blood glycosylated haemoglobin (HbAlc) was also measured by routine kit (Alfabiotech, Milano, Italy) using the autoanalyzer.

After rats were sacrificed, both eyes were either (1) enucleated and frozen at −80° C. for the measurements of the target biomarker(s) and/or other analysis or (2) are examined immediately post-sacrifice for morphological changes. For Western blot analyses protein extraction was performed by homogenizing the retina in 1 ml ice-cold hypotonic buffer A, containing 10 mM 2-[4-(2-Hydroxyethyl)-1-piperazinyl]ethanesulfonic acid [HEPES] (pH 7.8), 10 mM KCl, 2 mM MgCl2, 1 mM DTT, 0.1 mM EDTA, and 0.1 mM phenylmethylsulfonyl-fluoride (PMSF). The homogenates were added with 80 μl of 10% Nonidet P-40 (NP-40) solution and then centrifuged at 14,000×g for 2 min. The precipitates were washed once with 500 μl of buffer A plus 40 μl of 10% NP-40, centrifuged, re-suspended in 200 μl of buffer C [50 mM HEPES [pH 7.8], 50 mM KCl, 300 mM NaCl, 0.1 mM EDTA, 1 mM dihiothreitol [DTT], 0.1 mM PMSF, 20% glycerol], and recentrifugedat 14,800×g for 5 min. The supernatants were collected for determinations of GLUT-1, GLUT-3 and insulin according to the method described by Sahin et al.

Equal amounts of protein (50 μg) were electrophoresed and subsequently transferred onto a nitrocellulosemembrane (Schleicher and Schuell Inc., Keene, N.H., USA). Antibodies against target biomarker(s) were diluted as necessary in the same buffer containing 0.05% Tween-20. Protein loading was controlled sing a monoclonal mouse antibody against β-actin (A5316; Sigma). Bands were analyzed densitometrically using an image analysis system (Image J; National Institute of Health, Bethesda, USA).

After the eye extirpation, tissue (retina) of each rat was also examined grossly. The tissue was removed for histologic study, washed with normal saline, and immersion-fixed in 10% buffered formalin immediately upon removal. They were gradually dehydrated, embedded in paraffin, cut into 5-μm sections, and stained with hematoxylin and eosin for histologic examination according to standard procedures.

Data were analyzed statistically using one-way ANOVA. In the analyses for the biomarker(s), the repeated statement was added in the general linear model. The group differences were attained by the Fisher's multiple comparison test [Statistical Package for the Social Sciences (SPSS)]. A P value of less than 0.05 was considered significant.

### Example 1

### Chromium Histidinate Reduces Levels of Biomarkers Related to Diabetic Retinopathy in Rats

General procedures were conducted as described above. After rats were sacrificed, both eyes were enucleated and frozen at −80° C. for the measurements of MDA, GLUT 1, GLUT3 and insulin. The retina MDA content was measured by high performance liquid chromatography (HPLC, Shimadzu, Tokyo, Japan) using a Shimadzu UV-vis SPD-10 AVP detector and a CTO-10 AS VP column in a mobile phase consisting of 30 mM KH₂PO₄ and methanol (82.5+17.5, v/v; pH 3.6) at a flow rate of 1.2 ml/min. Column effluents were monitored at 250 nm and the volume was 20 μl. The retina homogenate (10%, w/v) was prepared in 10 mM phosphate buffer (pH 7.4), centrifuged at 13,000×g for 10 min at 4° C., and the supernatant was collected and stored at −80° C. for MDA analysis.

STZ administration affected the levels of typical blood parameters characteristic for diabetes, which are also accepted values in diabetes diagnostic (glucose, insulin and HbAlc). Blood glucose, HbAlc and total cholesterol levels were significantly increased in untreated diabetic rats compared to control groups while insulin levels were decreased (P<0.5). When diabetic retinopathy rats were treated with CrHis, significant increases in blood glucose, HbAlc, insulin and total cholesterol levels were observed in diabetic retinopathy rats. CrHis treatment also resulted in a significant decrease in mean serum total cholesterol concentration of diabetic retinopathy animals. Body weight was significantly decreased (P<0.001) in the untreated diabetic rats when compared to control group. CrHis treatment significantly increased body weight (P<0.001) compared to the untreated diabetic group (Table 1).

TABLE 1
Effect of CrHis supplementation on biochemical parameters in diabetic rats

| Parameters | Control | | CrHis | | STZ | | STZ + CrHis | |
|---|---|---|---|---|---|---|---|---|
| Body weight (g) | 330 | (3.6)bc | 340 | (3.9)b | 225 | (3.5)c | 250 | (3.5)a |
| Glucose (mg/dL) | 110 | (2.3)a | 100 | (2.0)a | 480 | (8.0)b | 290 | (2.7)c |
| Insulin (μU/mL) | 47.4 | (0.20)a | 50.3 | (0.22)a | 20.2 | (0.25)b | 25.0 | (0.25)c |
| Total cholesterol (mg/dL) | 90 | (0.64)a | 80 | (0.34)a | 240 | (0.90)b | 218 | (0.80)c |
| Glycosylated hemoglobin | 0.29 | (0.02)a | 0.20 | (0.01)a | 0.82 | (0.05)b | 0.45 | (0.03)c |
| (mg/g) | | | | | | | | |

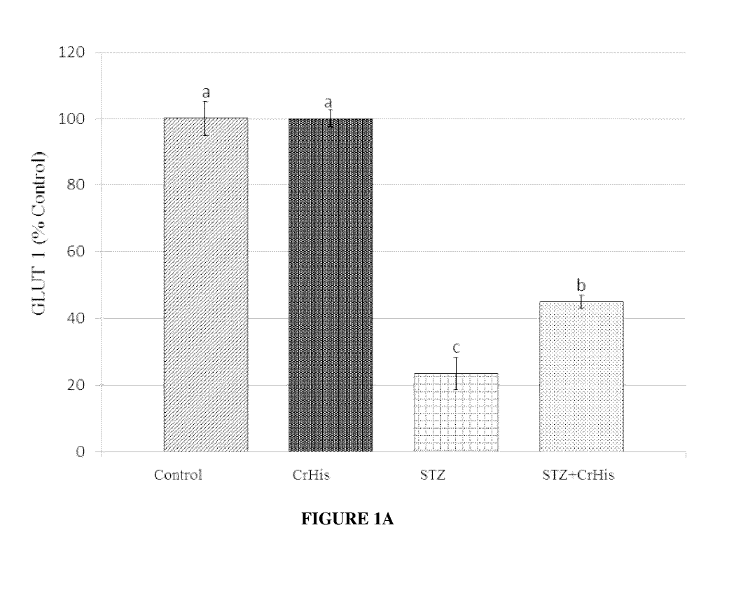

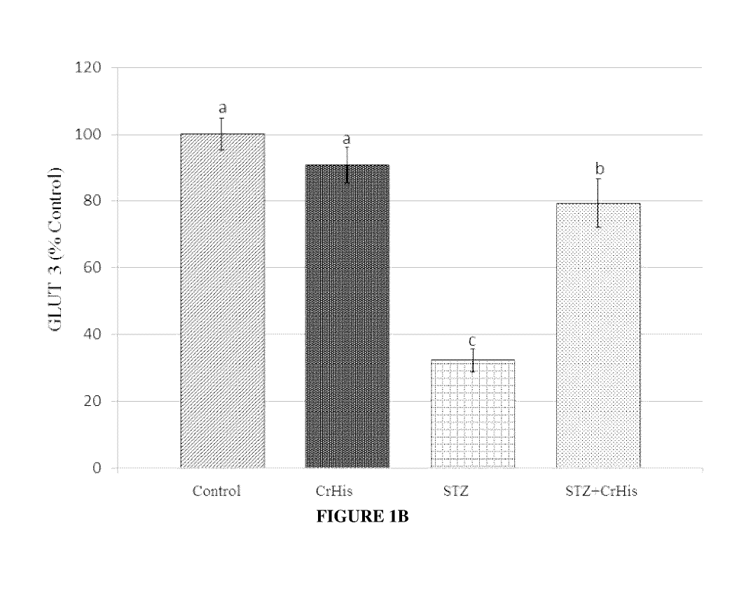

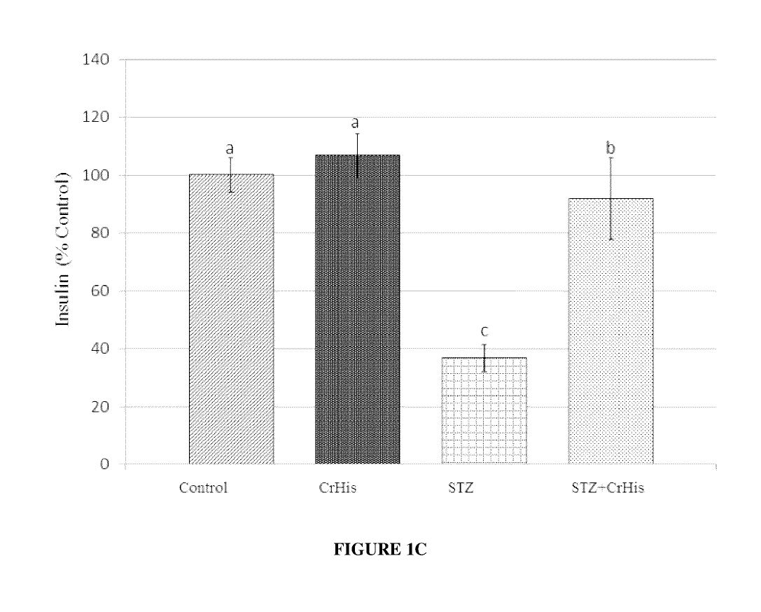

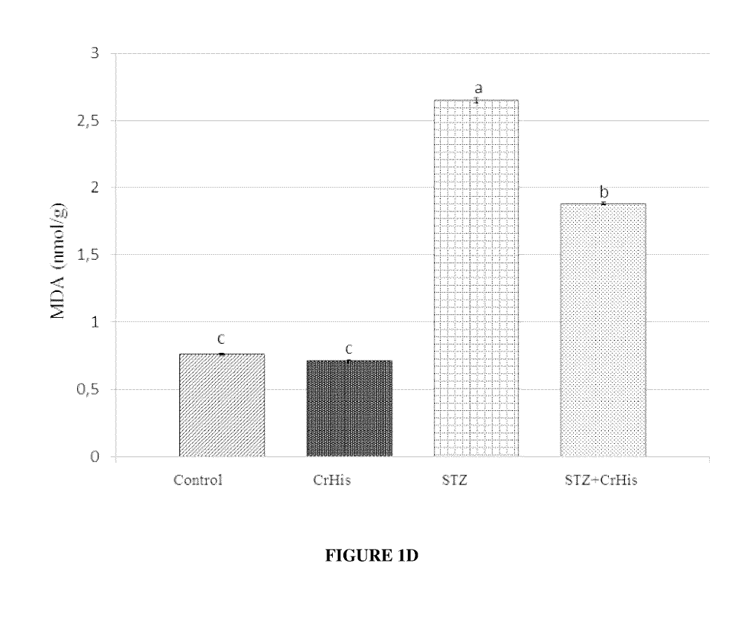

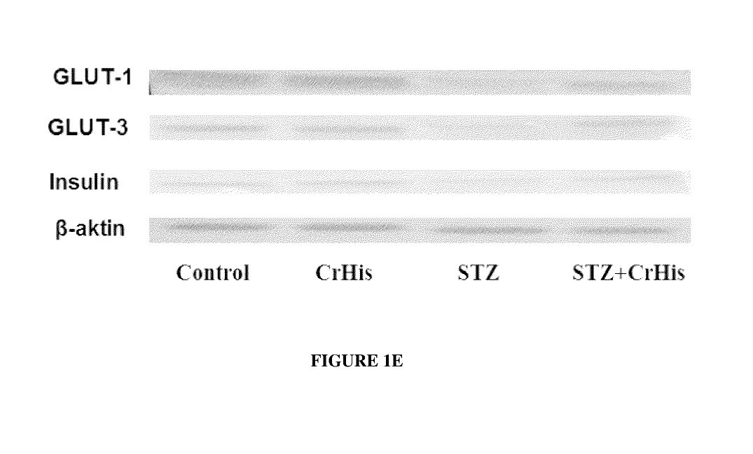

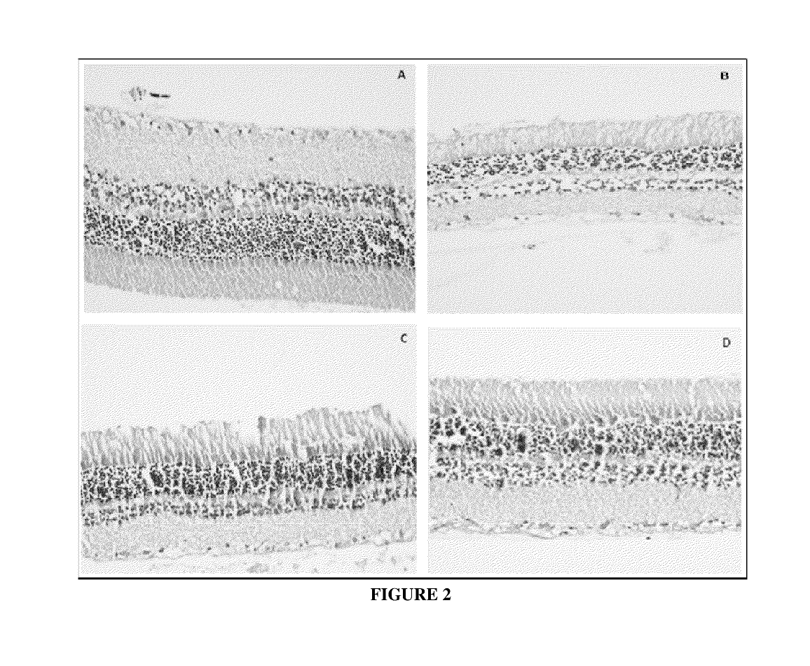

Expressions of GLUT1, GLUT3 and insulin showed significant upward regulation (P<0.05) in the retina of diabetic rats compared to control. Treatment using CrHis significantly (P<0.05) reversed these changes to near control levels (FIG. 1, Panel A-C).

Data are means of quadruplets of assays and expressed as relative to control (%). Blots were repeated at least 3 times (n=3) and a representative blot for each is shown. Actin was included to ensure equal protein loading. Values are LS means±SE. Different letters within the retina parts indicate statistical differences among groups (p<0.05).

The retina of untreated diabetic rats had considerably higher MDA expressions compared with controls (P<0.001). A statistically significant reduction of MDA expression was found in retina of diabetic rats when the diabetic rats were treated with CrHis (FIG. 1, Panel D).

### Example 2

### Chromium Histidinate Ameliorates the Physiological Effects of Diabetic Retinopathy Better than Other Chromium Species

General procedures were conducted as described above. Retinas were highly organized in the normal (control) rats, with intact layers. The retinas were disorganized in the diabetic rats with impaired layers. But the retinas in CrHis group were surprisingly improved compared to the diabetes group. Similar experiments are conducted with other chromium species such as chromium nicotinate and chromium picolinate. Surprisingly, the chromium histidinate complex is more efficacious at reducing the physiological effects of diabetic retinopathy than other chromium complexes at equivalent total dosages of chromium.

### Example 3

### Chromium Histidinate Reduces Retinal Lipid Oxidation

General procedures are conducted as described above. Retinal lipids are extracted from the retinal cellular lysate and characterized by liquid chromatography-mass spectrometry. Surprisingly, the chromium histidinate complex is more efficacious at reducing the retinal lipid oxidation than other chromium complexes at equivalent total dosages of chromium.

### Example 4

### Chromium Histidinate Reduces Free Radical Oxidation of Retinal Photoreceptors

General procedures are conducted as described above. Retinal lipids are extracted from the retinal cellular lysate and characterized by liquid chromatography-mass spectrometry. Surprisingly, the chromium histidinate complex is more efficacious at reducing the free radical oxidation of retinal photoreceptors than other chromium complexes at equivalent total dosages of chromium.

### Example 5

### Chromium Histidinate Reduces Both Hemoglobin Glycation and Retinal Protein Glycation

General procedures are conducted as described above. General procedures are conducted as described above. Retinal proteins are extracted from the retinal cellular lysate and characterized by liquid chromatography-mass spectrometry and/or Western Blotting. Surprisingly, the chromium histidinate complex is more efficacious at reducing hemoglobin glycation and/or retinal protein glycation than other chromium complexes at equivalent total dosages of chromium.

### Example 6

### Chromium Histidinate Decreases Loss of Retinal Lipoprotein Membrane Content

General procedures are conducted as described above. Retinal lipids are extracted from the retinal cellular lysate and characterized by liquid chromatography-mass spectrometry. Surprisingly, the chromium histidinate complex is more efficacious at decreasing the loss of retinal lipoprotein membrane content than other chromium complexes at equivalent total dosages of chromium.

### Example 7

### Chromium Histidinate Prevents Retinal Capillary Basement Membrane from Thickening

General procedures are conducted as described above. Retinal capillary basement membrane thickening is characterized by physical examination of the eye using standard techniques. Surprisingly, the chromium histidinate complex is more efficacious at preventing retinal capillary basement membrane from thickening than other chromium complexes at equivalent total dosages of chromium.

### Example 8

### Chromium Histidinate Decreases Microangiopathy

General procedures are conducted as described above. Retinal microangiopathy is characterized using standard ophthalomogic techniques. Surprisingly, the chromium histidinate complex is more efficacious at decreasing microangiopathy than other chromium complexes at equivalent total dosages of chromium.

### Example 9

### Chromium Histidinate Reduces Retinal Hard Exudates

General procedures are conducted as described above. Retinal hard exudates are extracted from the retinal cellular lysate and characterized by liquid chromatography-mass spectrometry. Surprisingly, the chromium histidinate complex is more efficacious at reducing hard exudates than other chromium complexes at equivalent total dosages of chromium.

### Example 10

### Co-administration of Chromium Histidinate with Insulin or Metformin Provides a Synergistic Effect in Treating Diabetic Retinopathy

General procedures are conducted as described above. Co-administration of chromium histidinate with insulin or metformin is performed. Retinal biomarker(s) are extracted from the retinal cellular lysate and characterized by liquid chromatography-mass spectrometry. Surprisingly, the results demonstrate that co-administration of chromium histidinate with insulin or metformin provides a synergistic effect in treating diabetic retinopathy than at equivalent total dosages of chromium and/or insulin metformin.

### Example 11

### Ophthalmic Solution

To prepare a pharmaceutical ophthalmic solution composition, 100 mg of chromium and histidine, chromium histidinate complexes, chromium trihistidinate, chromium polyhistidinate complexs, or combinations thereof, including pharmaceutically acceptable salts, hydrates, solvates, or mixtures thereof are mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

Each of the papers and patents discussed herein are expressly incorporated by reference in their entirety, including any drawings or figures.

What is claimed is:

1. A method for treating, preventing, or ameliorating diabetic retinopathy in a subject in need thereof, the method comprising:
identifying a subject having or at risk for developing diabetic retinopathy; and
administering a therapeutically effective amount of at least one chromium complex.

2. The method of claim 1, wherein administering comprises injecting at least one chromium complex into the subject.

3. The method of claim 1, wherein administering comprises injecting at least one chromium complex into an eye of the subject.

4. The method of claim 1, wherein the therapeutically effective amount of at least one chromium complex is administered orally.

5. The method of claim 1, wherein the at least one chromium complex consists essentially of chromium and histidine, a chromium histidinate complex, or combinations thereof.

6. The method of claim 1, wherein the at least one chromium complex is co-administered with a second therapeutic agent selected from the group consisting of insulin, metformin, and a chromium-insulin complex.

7. The method of claim 5, wherein the second therapeutic agent is administered orally.

8. The method of claim 1, wherein the administering a therapeutically effective amount of at least one chromium complex comprises administering a topical ophthalmic formulation.

9. A composition for topical ophthalmic administration comprising a therapeutically effective amount of one or more chromium complexes and at least one ophthalmically acceptable excipient.

10. A method of decreasing malondialdehyde levels in the eye of subject in need thereof, the method comprising:
identifying a subject having or at risk for developing increased malondialdehyde levels; and
administering a therapeutically effective amount of at least one chromium complex.

11. The method of claim 10, wherein the therapeutically effective amount of at least one chromium complex is topically administered to the eye.

12. The method of claim 10, wherein the therapeutically effective amount of at least one chromium complex is injected into the eye.

13. The method of claim 10, wherein the therapeutically effective amount of at least one chromium complex is administered orally.

14. The method of claim 10, wherein the at least one chromium complex consists essentially of chromium and histidine, a chromium histidinate complex, or combinations thereof.

15. The method of claim 10, wherein the at least one chromium complex is co-administered with a second therapeutic agent selected from the group consisting of insulin, metformin, and a chromium-insulin complex.

16. The method of claim 15, wherein the second therapeutic agent is administered orally.

17. The method of claim 10, wherein the administering a therapeutically effective amount of at least one chromium complex comprises administering a topical ophthalmic formulation.

18. A method of improving visual acuity in a subject having diabetic retinopathy, the method comprising:
identifying a subject having diabetic retinopathy; and
administering a therapeutically effective amount of at least one chromium complex.

19. The method of claim 18, wherein the at least one chromium complex consists essentially of chromium and histidine, a chromium histidinate complex, or combinations thereof.

20. The method of claim 18, wherein the at least one chromium complex is co-administered with a second therapeutic agent selected from the group consisting of insulin, metformin, and a chromium-insulin complex.
